(19) United States
(12) Patent Application Publication
(10) Pub. No.: US 2002/0136744 A1
(43) Pub. Date: Sep. 26, 2002

(54) Sustained release drug dispersion delivery device

(75) Inventors: Michael W. McGlynn, Jenkintown, PA (US); Mandana Asgharnejad, Ambler, PA (US)

Correspondence Address: MERCK AND CO INC, P O BOX 2000, RAHWAY, NJ, 070650907

(73) Assignee: Merck & Co., Inc.

(21) Appl. No.: 10/096,212

(22) Filed: Mar. 12, 2002

Related U.S. Application Data
(63) Continuation of application No. 09/616,508, filed on Jul. 14, 2000.
(60) Provisional application No. 60/144,643, filed on Jul. 20, 1999.

(51) Int. Cl. A61K 9/00; A61K 31/497

(52) U.S. Cl. 424/400; 514/252.1

(57) ABSTRACT

The present invention is related to a drug delivery device, that is pH insensitive, for the sustained in situ production and release of a dispersion, in an environment of use, which comprises

a) a compressed core prepared from an admixture comprising

i) a therapeutically effective amount of a beneficial agent that has a solubility profile that is dependent on the pH level of the environment of use;

ii) a water swellable polymer which upon hydration forms gelatinous microscopic particles; and

iii) a pH modulator; and

b) a water insoluble, water impermeable polymeric coating comprising a polymer and a plasticizer, which surrounds and adheres to the compressed core, said water insoluble, water impermeable polymeric coating having at least one aperture.

### BACKGROUND OF THE INVENTION

The need for systems that can deliver any drug at a controlled rate of release to an environment of use over a specified period of time is well established.

U.S. Pat. No. 4,814,182 discloses the use of rods or slabs of pre-hydrated and swelled polyethylene oxide hydrogel. The polymer is impregnated with a biologically active agent during the hydration procedure. The hydrated polymer is then dried and partially coated with an impermeable, insoluble material. When placed in an aqueous environment, the polymer swells but does not dissolve or disintegrate. The entrapped active ingredient is released form the polymer by diffusion. The mechanism of release is based on the ability of the soluble drug to diffuse through the rehydrated hydrogel and move into the aqueous environment.

U.S. Pat. No. 4,839,177 discloses the use of hydrogels compressed to defined geometric forms. In this device, the polymer is mixed with biologically active ingredients to form a core which is affixed to a “support platform” made of an insoluble polymeric material. When hydrated, the swellable, gellable hydrogel expands beyond the device and establishes a superstructure from which the active agent is release either by diffusion, if the active agent is soluble, or by erosion, if the active agent is insoluble. The generation and maintenance of the superstructure is vital to the proper operation of this device.

An osmotic dosage form which utilizes a semipermeable wall containing at least one “exit means” which passes through the wall, surrounding a core containing an osmotic agent, a neutral and ionizable hydrogel and an active ingredient is taught in U.S. Pat. No. 4,971,790. The coating of this device is permeable to water from the environment of use. Water moves into the core through the semipermeable membrane. Once inside the device, the water solubilizes the osmotic agent, and hydrates the hydrogels. Pressure builds up inside the device. Ultimately, the solubilized hydrogel, containing the beneficial agent, and other core excipients are pumped out of the core, under pressure, through an exit means and into the environment of use.

The existing technology is limited since diffusion controlled systems are effective only when soluble agents are dispensed. For osmotically controlled devices, the technology relies upon a wall permeable to the passage of fluid present in the environment of use. Furthermore, these devices require a wall of carefully controlled permeability.

Devices which rely upon the establishment of an extra device superstructure can be altered during in vivo transit, for example, in the gastrointestinal tract. If portions of the superstructure break away, greater surface area is exposed to the environment and unpredictable release of the active agent may result.

U.S. Pat. No. 5,366,738 discloses a device and method that improves the delivery of drugs. This device and method avoids the diffusion from a swelled polymer or through the superstructure of a polymeric matrix. U.S. Pat. No. 5,366,738 also discloses a device where the generation of an extra tablet structure could be avoided and the dry ingredients can be contained within a protective coating until released from the device. This prevents the chance of premature erosion and uncontrolled release of the active agent as well as provides enhanced stability for those active agents that are labile in the fluid of the environment of use.

A frequently encountered problem in the field of sustained release compositions is that many water-miscible drugs have a tendency to be dumped or surged into the body during the first hour or two after an oral dosage form is ingested. This problem is particularly acute when the sustained release compositions are administered with food. Several U.S. Pat. Nos. 4,789,549, 4,816,264 and 4,851,233, have disclosed devices that have an improved sustained release activity. However, none are entirely satisfactory since they have a tendency to rapidly release water-miscible drugs when administered with food. Additionally, the devices disclosed are not insensitive to the pH of the environment of use.

It would be useful to have a device where the mechanism of release is insensitive to the pH level of the environment of use. Such a device is particularly important for cancer patients since such patients may have metabolic or other gastrointestinal problems or abnormalities.

It is, therefore, an object of this invention to develop a sustained release drug dispersion delivery device, which has a mechanism of release that is insensitive to the pH level of the environment of use, for a drug with a pH-dependent solubility profile.

It is also an object of this invention to develop a sustained release drug dispersion delivery device that contains a pH modulator which maintains the pH of the delivery device at a sufficient level to allow the beneficial agent to remain insoluble until its release into the environment of use and assists in the release of the beneficial agent into the environment of use.

It is also an object of this invention to develop a sustained release drug dispersion delivery device that ensures the continuous delivery of a beneficial agent and avoids the possibility of dose dumping.

### SUMMARY OF THE INVENTION

The present invention is related to a drug delivery device, that is pH insensitive, for the sustained in situ production and release of a dispersion, in an environment of use, which comprises

a) a compressed core prepared from an admixture comprising

i) a therapeutically effective amount of a beneficial agent that has a solubility profile that is dependent on the pH level of the environment of use;

ii) a water swellable polymer which upon hydration forms gelatinous microscopic particles; and

iii) a pH modulator; and

b) a water insoluble, water impermeable polymeric coating comprising a polymer and a plasticizer, which surrounds and adheres to the compressed core, said water insoluble, water impermeable polymeric coating having at least one aperture.

### BRIEF DESCRIPTION OF THE DRAWINGS

FIG. 1 depicts the release rates of the beneficial agent without a pH modulator present in the instant invention.

FIG. 2 depicts the release rates of the beneficial agent where a pH modulator is present in the instant invention.

### DETAILED DESCRIPTION OF THE INVENTION

The present invention is related to a drug delivery device, that is pH insensitive, for the sustained in situ production and release of a dispersion, in an environment of use, which comprises

a) a compressed core prepared from an ad-mixture comprising

i) a therapeutically effective amount of a beneficial agent that has a solubility profile that is dependent on the pH level of the environment of use;

ii) a water swellable polymer which upon hydration forms gelatinous microscopic particles; and

iii) a pH modulator; and

b) a water insoluble, water impermeable polymeric coating comprising a polymer and a plasticizer, which surrounds and adheres to the compressed core, said water insoluble, water impermeable polymeric coating having at least one aperture.

The instant invention provides a means for administering, in a sustained-release manner up to about a 24 hour period, a therapeutic dose of a beneficial agent that has a water solubility profile that is highly dependent on pH levels in the environment of use. Preferably, the beneficial agent is released over about a 4 to about a 12 hour period. More preferably, the beneficial agent is released over about a 6 to about an 8 hour period. This invention is particularly useful for beneficial agents which are very soluble at low pH values (less than about 2) and are practically insoluble at near-neutral pH values (greater than or equal to about 5) ensuring a sustained release of the beneficial agent throughout all pH values. In one embodiment of the instant invention, the preferred beneficial agent is 1-(3-chlorophenyl)-4-[1-(4-cyanobenzyl)-5-imidazolyl methyl]-2-piperazinone, which has a solubility profile that is highly dependent on pH levels, in that it is very soluble at low pH levels (less than about 2) and practically insoluble at near neutral pH levels (greater than about 5).

Another embodiment of this instant invention is directed to a process for the preparation of the drug delivery device, that is pH insensitive, for the sustained in situ production and release of a beneficial agent comprising:

a) preparing the compressed core by either dry or wet granulation of the swellable polymer, the medicament and other excipients required in the preparation of tablets and compressing the mixture into cores;

b) coating the entire core with the coating material; and

c) putting apertures through the coating using mechanical, laser-based, or ultrasonic excitation techniques.

In one embodiment of the instant invention, a tablet, comprising the compressed core and water insoluble, water impermeable polymeric coating, is formed. This tablet is laser drilled to create a plurality of apertures which penetrate the coating. The apertures allow for the flow of liquids between the environment of use and the compressed core of the tablet. The liquids present in the environment of use flow into the core and dissolve the pH modulator. The pH modulator will begin to neutralize the compressed core by elevating the pH levels in the compressed core. Upon hydration by the liquids, the water soluble polymer will begin to swell. The swelling of the polymer results in the release of the beneficial agent, or active compound, into the environment of use via a gel extrusion mechanism. While the pH modulator regulates the degree and rate of swelling by maintaining a high pH level, the size and number of apertures in the coating will regulate the dispersion rate of the beneficial agent. Because the pH level in the core is near neutral, the beneficial agent remains insoluble and avoids the possibility of “dose dumping” in the stomach. Under these conditions, the beneficial agent is released by the gel extrusion mechanism and dissolution/diffusion of the beneficial agent does not occur. Thus the instant invention can achieve a sustained-release of the beneficial agent, preferably over about a 6 to about an 8 hour period of time.

By “drug delivery device” is meant, a dosage form that provides a convenient means of delivering a drug to a subject. The subject can be a human or any other animal. The device is designed to be useful for the delivery of a drug by any pharmaceutically accepted means such as by swallowing, retaining it within the mouth until the beneficial agent has been dispensed, placing it within the buccanal cavity, or the like.

By “sustained” production is meant that the rate of release of the beneficial agent, that is the amount of beneficial agent released from the device to the environment of use, follows a predetermined pattern. Thus, relatively constant or predictably varying amounts of the beneficial agent can be dispensed over a specified period of time.

By “compressed core” is meant an admixture of ingredients comprising a beneficial agent, a water swellable polymer which produces gelatinous microscopic particles when hydrated, a pH modulator and other ingredients that may affect any of: (1) the rate of production of the dispersion; (2) the stability of the components of the dosage form; or (3) the mixing or compression characteristics of the admixture, is blended in such a way to produce a uniform material. This uniform material is then compressed, within a die, to produce a desired form, normally in the shape of a tablet, capsule or bolus.

The term “beneficial agent” broadly includes any drug or mixture thereof, that can be delivered from the system to produce a beneficial result. In the specification and the accompanying claims, the term “beneficial agent”, “drug” or their equivalents include any physiologically or pharmacologically active substance that produces a localized or systemic effect or effects in animals. The term “animal” includes mammals, humans and primates such as domestic household, sport or farm animals such as sheep, goats, cattle, horses and pigs; laboratory animals such as mice, rats and guinea pigs, fish, avians, reptiles and zoo animals.

The beneficial agent that can be delivered by the novel device of this invention, includes inorganic and organic compounds without limitation, including drugs that act on the peripheral nerves, adrenergic receptors, cholinergic receptors, nervous system, skeletal muscles, cardiovascular system, smooth muscles, blood circulatory system, synaptic sites, neuroeffector junctional sites, endocrine and hormone systems, immunological system, reproductive system, skeletal systems, autocoid systems, alimentary and excretory systems, inhibitory and histamine systems, and those materials that act on the central nervous system such as hypnotics and sedatives.

Examples of beneficial drugs are disclosed in Remington's Pharmaceutical Sciences, 16th Ed., 1980, published by Mack Publishing Co., Eaton, Pa.; and in The Pharmacological Basis of Therapeutics, by Goodman and Gilman, 6th Ed., 1980, published by the MacMillan Company, London; and in The Merck Index, 11th Edition, 1989, published by Merck & Co., Inc., Rahway, N.J. Specific examples of beneficial agents, or drugs, that may be adapted for use include prenyl protein inhibitors, particularly farnesyl-protein transferase inhibitors, such as those disclosed in the following patents, pending applications and publications, which are herein incorporated by reference:

U.S. Pat. No. 5,238,922 issued on Aug. 24, 1993;

U.S. Pat. No. 5,340,828, issued on Aug. 23, 1994;

U.S. Pat. No. 5,480,893 issued on Jan. 2, 1996;

U.S. Pat. No. 5,352,705 issued on Oct. 4, 1994;

U.S. Pat. No. 5,504,115 issued on Apr. 2, 1996;

U.S. Pat. No. 5,326,750 issued on Jul. 16, 1994;

U.S. Pat. No. 5,504,212 issued on Apr. 2, 1996;

U.S. Pat. No. 5,686,472 issued on Nov. 11, 1997;

U.S. Pat. No. 5,736,539 issued on Apr. 7, 1998;

U.S. Pat. No. 5,439,918 issued on Aug. 8, 1995;

U.S. Pat. No. 5,576,313 issued on Nov. 19, 1996;

U.S. Pat. No. 5,571,835 issued on Nov. 5, 1996;

U.S. Pat. No. 5,491,164 issued on Feb. 13, 1996;

U.S. Pat. No. 5,631,280 issued on May 20, 1997;

U.S. Pat. No. 5,576,293 issued on Nov. 19, 1996;

U.S. Pat. No. 5,468,733 issued on Nov. 21, 1995;

U.S. Pat. No. 5,585,359 issued on Dec. 17, 1996;

U.S. Pat. No. 5,523,456 issued on Jun. 4, 1996;

U.S. Pat. No. 5,652,257 issued on Jul. 29, 1997;

U.S. Pat. No. 5,661,161 issued on Aug. 26, 1997;

U.S. Pat. No. 5,578,629 issued on Nov. 26, 1996;

U.S. Pat. No. 5,627,202 issued on May 6, 1997;

U.S. Pat. No. 5,624,936 issued on Apr. 29, 1997;

U.S. Pat. No. 5,534,537 issued on Jul. 9, 1996;

U.S. Pat. No. 5,710,171 issued on Jan. 20, 1998;

U.S. Pat. No. 5,703,241 issued on Dec. 30, 1997;

U.S. Pat. No. 5,856,326 issued on Jan. 5, 1999;

U.S. Pat. No. 5,710,171 issued on Jan. 20, 1998;

U.S. Pat. No. 5,756,528 issued on May 26, 1998;

U.S. Ser. No. 08/960,248, filed on Oct. 29, 1997;

U.S. Pat. No. 5,817,678 issued on Oct. 6, 1998;

U.S. Ser. No. 08/786,516, filed on Jan. 21, 1997;

U.S. Ser. No. 08/786,520, filed on Jan. 21, 1997; U.S. Ser. No. 09/015,283, filed on Jan. 29, 1998;

U.S. Ser. No. 08/784,556, filed on Jan. 21, 1997; U.S. Ser. No. 09/030,223, filed on Feb. 25, 1998;

U.S. Ser. No. 08/786,519, filed on Jan. 21, 1997;

U.S. Ser. No. 08/823,921, filed on Mar. 25, 1997;

U.S. Pat. No. 5,859,012, issued on Jan. 12, 1999;

U.S. Ser. No. 08/834,671, filed on Apr. 1, 1997;

U.S. Ser. No. 08/827,485, filed on Mar. 27, 1997;

U.S. Pat. No. 5,852,010 issued on Dec. 22, 1998;

U.S. Ser. No. 08/823,920, filed on Mar. 25, 1997; U.S. Ser. No. 09/164,741, filed on Oct. 10, 1998;

U.S. Pat. No. 5,780,488 issued on Jul. 14, 1998;

U.S. Pat. No. 5,859,015 issued on Jan. 12, 1999;

U.S. Ser. No. 08/824,427, filed on Mar. 26, 1997;

U.S. Pat. No. 5,780,492 issued on Jul. 14, 1998;

U.S. Pat. No. 5,891,889, issued on Apr. 6, 1999;

U.S. Pat. No. 5,885,995 issued on Mar. 23, 1999;

U.S. Ser. No. 08/823,934, filed on Mar. 25, 1997;

U.S. Ser. No. 08/834,675, filed on Apr. 1, 1997;

U.S. Ser. No. 08/823,929, filed on Mar. 25, 1997;

U.S. Pat. No. 5,869,682 issued on Feb. 9, 1999;

U.S. Ser. No. 08/823,919, filed on Mar. 25, 1997;

U.S. Pat. No. 5,859,035 issued on Jan. 12, 1999;

U.S. Pat. No. 5,854,264 issued on Dec. 29, 1998;

U.S. Pat. No. 5,833,105 issued on Mar. 16, 1999;

U.S. Pat. No. 5,854,265 issued on Dec. 29, 1998;

U.S. Ser. No. 08/829,922, filed on Apr. 1, 1997;

U.S. Pat. No. 5,874,452 issued on Feb. 23, 1999;

U.S. Pat. No. 5,880,140 issued on Mar. 9, 1999;

U.S. Pat. No. 5,872,136 issued on Feb. 16, 1999;

U.S. Ser. No. 08/984,732, filed on Dec. 4, 1997;

U.S. Ser. No. 08/985,124, filed on Dec. 4, 1997;

U.S. Ser. No. 08/985,337, filed on Dec. 4, 1997;

U.S. Ser. No. 08/985,320, filed on Dec. 4, 1997;

U.S. Ser. No. 08/995,744, filed on Dec. 22, 1997;

U.S. Ser. No. 08/997,171, filed on Dec. 23, 1997;

U.S. Ser. No. 09/170,952, filed on Oct. 13, 1998;

U.S. Ser. No. 09/167,180, filed on Oct. 6, 1998;

U.S. Ser. No. 09/332,769, filed on Jun. 14, 1999;

U.S. Ser. No. 09/164,482, filed on Oct. 1, 1998;

U.S. Ser. No. 09/140,919, filed on Aug. 26, 1998;

U.S. Ser. No. 09/140,584, filed on Aug. 26, 1998;

U.S. Ser. No. 09/195,578, filed on Nov. 19, 1998;

U.S. Ser. No. 09/342,701, filed on Jun. 29, 1999;

U.S. Ser. No. 60/122,968, filed on Mar. 3, 1999; and U.S. Ser. No. 60/127,132, filed on Mar. 31, 1999;

U.S. Ser. No. 60/122,970, filed on Mar. 3, 1999; and U.S. Ser. No. 60/127,259, filed on Mar. 31, 1999;

U.S. Ser. No. 60/122,768, filed on Mar. 3, 1999; and U.S. Ser. No. 60/127,253, filed on Mar. 31, 1999;

U.S. Ser. No. 60/122,771, filed on Mar. 3, 1999; and U.S. Ser. No. 60/127,257, filed on Mar. 31, 1999;

U.S. Ser. No. 60/123,620, filed on Mar. 3, 1999; and U.S. Ser. No. 60/127,252, filed on Mar. 31, 1999;

U.S. Ser. No. 60/111,416, filed on Dec. 8, 1998, U.S. Ser. No. 60/129,282, filed on Apr. 14, 1999; and

U.S. Ser. No. 60/111,621, filed on Dec. 8, 1998.

The following compounds, which are inhibitors of farnesyl-protein transferase, may also be adapted for use in the instant invention described herein:

(+)-6-[amino(4-chlorophenyl)(1-methyl-1H-imidazol-5-yl)methyl]-4-(3-chlorophenyl)-1-methyl-2(1H)-quinolinone (Compound J)

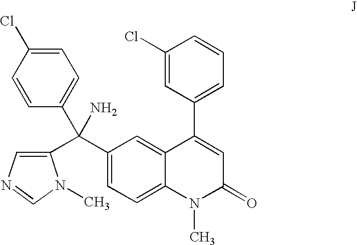

(−)-6-[amino(4-chlorophenyl)(1-methyl-1H-imidazol-5-yl)methyl]-4-(3-chlorophenyl)-1-methyl-2(1H)-quinolinone (Compound J-A; designated “comp. 74” in WO 97/21701); (+)-6-[amino(4-chlorophenyl)(1-methyl-1H-imidazol-5-yl)methyl]-4-(3-chlorophenyl)-1-methyl-2(1H)-quinolinone (Compound J-B; designated “comp. 75” in WO 97/21701) or a pharmaceutically acceptable salt thereof. The syntheses of these compounds are specifically described in PCT Publication WO 97/21701, in particular on pages 19-28. The preferred compound among these compounds to use in the instant formulation is Compound J-B. Other compounds described in PCT Publication WO 97/21701 may also be beneficially administered using the instant formulation.

The following compound which is an inhibitor of farnesyl-protein transferase may also be adapted for use in the instant invention described herein:

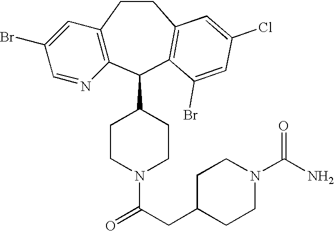

or a pharmaceutically acceptable salt thereof. The synthesis of this compound is specifically described in PCT Publication WO 97/23478, in particular on pages 18-56. In WO 97/23478, the above compound is designated compound “39.0” and is specifically described in Example 10. Other compounds described in PCT Publication WO 97/23478 may also be beneficially administered using the instant formulation.

All patents, publications and pending patent applications identified are herein incorporated by reference.

One particular beneficial agent that can be used in the instant invention is 1-(3-chlorophenyl)-4-[1-(4-cyanobenzyl)-5-imidazolyl methyl]-2-piperazinone, as described in U.S. Pat. No. 5,856,326, herein incorporated by reference. The above list of drugs is not meant to be exhaustive. Many other drugs will certainly work in the instant invention.

The dissolved drug can be in various forms, such as charged molecules, charged molecular complexes, ionizable salts or hydrates. Acceptable salts include, but are not limited to hydrochlorides, hydrobromide, sulfate, laurylate, palmitate, phosphate, nitrate, borate, acetate, maleate, malate, succinate, trimethamine, tartrate, oleate, salicylate, salts of metals, and amines or organic cations, for example quaternary ammonium.

Derivatives of drugs such as esters, ethers and amides without regard to their ionization and solubility characteristics can be used alone or mixed with other drugs. Also, a drug can be used in a form that, upon release from the device, is converted by enzymes, hydrolyzed by body pH or other metabolic processes to the original form, or to a biologically active form.

By “therapeutically effective amount” is meant that the quantity of beneficial agent contained in the core, which can be delivered to the environment of use, has been demonstrated to be sufficient to induce the desired effect during studies utilizing the beneficial agent.

The beneficial agent can be in the core as a dispersion, particle, granule or powder. Also, the beneficial agent can be mixed with a binder, dispersant, emulsifier or wetting agent and dyes.

The beneficial agent may comprise from about 0.01% to about 75% by weight of the core mixture. Generally, the device can house from about 0.05 ng to about 50 grams of beneficial agent or more, with individual devices containing, for example about 25 ng, about 1 mg, about 5 mg, about 250 mg, about 500 mg, about 1.5 g, about 5 g, or the like. Preferably, the device comprises about 1 mg to about 1 gram of beneficial agent. Most preferably, the device comprises about 5 mg to about 500 mg of beneficial agent.

The phrase “water swellable polymer which upon hydration forms gelatinous microscopic particles” broadly encompasses any polymer that upon hydration, is capable of producing discrete gelationous microscopic particles (“gel”) which support a dispersion, including the beneficial agent, as it forms. The gelatinous forming, water swellable polymer used also must move from the core surface in such a way that the beneficial agent is carried into the environment of use. Upon hydration, the hydrated gel is forced out of the compressed core due to the volume expansion of the polymer within the compressed core. This water swellable polymer is capable of swelling in water and/or in the gastric intestinal fluid.

Illustrative of this type of water swellable polymer are the superabsorbant polymers, such as sodium polyacrylate, particularly those compositions sold under the trade names, “AQUAKEEP® J-550”, “AQUAKEEP® J-400”, which are trade names for sodium acrylate polymer produced by Seitetsu Kagaku Co., Ltd, Hyogo, Japan. The “AQUAKEEP®” polymers are generically described in U.S. Pat. No. 4,340,706. Also illustrative of this type of water swellable polymer are carboxypolymethylenes prepared from acrylic acid crosslinked with allyl ethers of sucrose or pentaerythritol and sold under the trade names “CARBOPOL® 934P”, “CARBOPOL® 974P” and “CARBOPOL® 971P”, which are trade names for two carbomer type polymers produced by B. F. Goodrich Chemical Company, Cleveland, Ohio. These latter polymers are generically described in U.S. Pat. No. 2,909,462 and in the National Formulary XVII at page 1911, CAS Registry Number 9003-01-4. All of the foregoing references are hereby incorporated by reference. Also illustrative of this type of water swellable polymer is polyethylene oxide.

Additionally, the water swellable polymers that form usable gelatinous microscopic particles may include the pharmaceutically acceptable salts of the superabsorbant polymers such as AQUAKEEP® J550, AQUAKEEP® J400, CARBOPOL® 974P, CARBOPOL® 971P and CARBOPOL® 934P. By “pharmaceutically acceptable salts” of the polymers is meant the acid form of the polymer neutralized by converting all or a portion of the free acid functional groups to their salt form. The core of the device contains from about 5% to about 75%, by weight of the core mixture, of the dry gelatinous microscopic particle polymer. Preferably, the core device contains from about 5% to about 50%, by weight, of the dry gelatinous microscopic particle polymer. Most preferably, the core device contains from about 5% to about 30%, by weight, of the dry gelatinous microscopic particle polymer.

The “gelatinous microscopic particles” are composed of discrete particles of hydrated polymer. Both size and hydration rate of these gelatinous microscopic particles are characteristics of the individual polymers. In the dry state, CARBOPOL® 974P, CARBOPOL® 971P, and CARBOPOL® 934P particles range in size from about 2 to about 7 microns. When these particles are hydrated, gelatinous microscopic particles of about 20 microns are produced. When AQUAKEEP® J-550 or AQUAKEEP® J-400 particles are hydrated, the diameter of the gelatinous microscopic particles can range in size from about 100 to about 1000 microns.

Once the drug delivery device is within the environment of use, the water swellable polymer in the compressed core, which is exposed to the ambient aqueous solution at the coating apertures, begins to hydrate and produce gelatinous microscopic particles. By “in situ production and release of a dispersion” is meant that, during the production of the gelatinous microscopic particles, soluble and insoluble core components located near the polymer particles become dispersed and mixed in such a manner that a gelatinous dispersion is produced. The dispersion extrudes through the apertures of the device into the aqueous solvent, bringing the beneficial agent into the environment of use. In this novel device, the components of the compressed core move into the environment of use, carried along by the gelatinous microscopic particles, continually exposing new surfaces for further hydration and production of the dispersion.

By “gelatinous” is meant a semisolid system consisting of hydrated polymer interpenetrated by the aqueous solvent of the environment of use.

The “pH modulator” useful in the novel device of this invention broadly encompasses any water soluble compound that can inhibit or enhance the rate of hydration of the gelatinous forming polymer of the core. In the instant invention, the pH modulator maintains the pH level of the compressed core of the instant device at a sufficiently high value to allow the beneficial agent to remain insoluble and the water swellable polymer to swell and cause the release of the beneficial agent. Among the groups of compounds that can exert this effect are bases and the salts of bases such as sodium carbonate, sodium bicarbonate, betaine hydrochloride, sodium citrate, arginine, meglamine, sodium acetate, sodium phosphates, potassium phosphates, calcium phosphate, ammonium phosphate, magnesium oxide, magnesium hydroxide, sodium tartrate and tromethamine. Other compounds that can be used as polymer hydration modifiers include sugars such as lactose, sucrose, mannitol, sorbitol, pentaerythritol, glucose and dextrose. Polymers such as microcrystalline cellulose and polyethylene glycol, as well as surfactants and other organic and inorganic salts can also be used to modulate polymer hydration. Most preferably, sodium phosphate dibasic is used.

The pH modulating agents are solubilized by the aqueous media of the environment of use and establish an environment within the core such that the pH, ionic strength or hydrophilic character is appropriate for the desired polymer gelatinous microscopic particle hydration rate. For example, these pH modulating agents can enhance or retard the neutralization of acidic functional groups on the polymer which affects the rate of hydration.

Other excipients such as lactose, magnesium stearate, microcrystalline cellulose, starch, stearic acid, citric acid, ascorbic acid, BHA (Butylated Hydroxyanisole), calcium phosphate, glycerol monostearate, sucrose, polyvinylpyrrolidone, gelatin, methylcellulose, sodium carboxymethylcellulose, sorbitol, mannitol, polyethylene glycol and other ingredients commonly utilized as stabilizing agents or to aid in the production of tablets may also be present in the core.

The core compartment containing the beneficial agent, pH modulator, and water, swellable polymer, as described herein, is typically in the form of a solid conventional tablet. Generally, the core, or “core mixture”, is compressed into its final shape using a standard tablet compressing machine. The core may contain compressing aids and diluents such as microcrystalline cellulose and lactose, repectively, that assist in the production of compressed tablets. The core can be comprised of a mixture of agents combined to give the desired manufacturing and delivery characteristics. The number of agents that may be combined to make the core is substantially without an upper limit with the lower limit equaling three components: (1) the beneficial agent (or drug), (2) the water swellable polymer, and (3) the pH modulator.

The specifications for the core are summarized below and include:

1. Core Drug Loading (size): about 0.01% to about 75% by weight of the total core mixture or about 0.05 nanogram to about 50 grams or more (includes dosage forms for humans and animals);

2. pH modulator: about 1% to about 75% by weight of the total core mixture; and

3. Water Swellable Polymer: about 5% to about 75% by weight of the total core mixture.

More preferably, the pH modulator will comprise about 10% to about 65% by weight of the total core mixture. Most preferably, the pH modulator will comprise about 40% to about 55% by weight of the total core mixture.

In cases where the beneficial agent, the water swellable polymer and pH modulator exhibit the desired release rate, stability, and manufacturing characteristics, there is no critical upper or lower limit as to the amount of beneficial agent that can be incorporated into a core mixture. The ratio of beneficial agent to excipient is dictated by the desired time span and profile of release, and the pharmacological activity of the beneficial agent.

Generally, the core will contain 1% to 75% by weight of the core mixture, of a beneficial agent admixed with other solute(s). Representative of compositions of matter that can be released from the device and can function as a solute are, without limitation, those compositions as described.

The coating, applied to the compressed core, is a material that is impermeable and insoluble in the fluid of the environment of use, can form films, and does not adversely affect the drug, animal body, or host. The coating is impermeable to water and also impermeable to the selected product, drugs, polymer hydration modulating agents, or to other compounds in the device. This impermeable material is insoluble in body fluids and non-erodible or it can be bioerodible after a predetermined period with bioerosion following the end of the active drug release period. In each instance, it is impermeable to solvent and solute(s) and is suitable for construction of the device.

By “impermeable” is meant that the influx of water across the coating is de minimus. Flux of water into the device is via the apertures placed in the coating.

The polymeric coating is applied to and adheres to the entire surface of the core. Apertures are produced in the coating to expose the core, using either a drill, a laser, a coring device or any other pharmaceutically accepted means.

The apertures allow liquids from the environment of use to enter the compressed core and make contact with exposed portions of the core when in use. The number, size and configuration of the apertures is chosen to provide the release rate required to suit a pharmacologically recognized requirement.

The coating can be applied by dipping the cores into a solution of the polymer or by coating the cores using a pharmaceutically acceptable polymer coating process. The groups of polymers that can provide this type of protection include, but are not limited to, cellulose acetate, cellulose acetate butyrate, ethylcellulose, polyvinylacetate, polyvinyl chloride and polymers of acrylic and methacrylic acid esters. In addition, other materials, such as plasticizers, may be included with the coating to enhance its stability, color, elasticity, flexibility, ease of application or opacity. Types of plasticizers that may be used include, but are not limited to, dibutylsebacate, diethylphthalate, triethylcitrate and polyethylene glycol. Preferably, the polymer comprises polyvinyl chloride, cellulose acetate, cellulose acetate butyrate or ethylcellulose, or combinations thereof. Preferably, the plasticizer comprises diethylphthalate, dibutylsebacate or triethylcitrate.

The coating is applied to a thickness of from about 1 to about 1000 microns. Preferably, the thickness of the coating is about 10 to about 500 microns, although thinner and thicker coatings fall within the scope of the invention.

The expression “aperture” as used herein, refers to ports through the coating which expose the surface of the core to the environment. The size and number of apertures is chosen to effect the desired release rate. Exposure of from about 1% to about 75% of the core surface is contemplated by this invention. Preferably, the coating has a plurality of apertures exposing between about 1 and about 75% of the core surface, wherein the release rate of beneficial agent from the device is a function of the number and size of the apertures. More preferably, the coating has a plurality of apertures exposing between about 5 and about 50% of the core surface. Most preferably, the coating has a plurality of apertures exposing between about 8 and about 25% of the core surface. The apertures are generally positioned in a regular pattern on both faces of the device although they can be positioned anywhere on the core including the edges or all on one face.

The apertures are generally circular, but may be of any design that results in the proper release rate. When the aperture is circular, its diameter ranges from about 0.05 mm to about 20 mm. Preferably, the diameters of the aperture are about 0.1 mm to about 5 mm typical. Most preferably, the diameter ranges are about 0.2 mm to about 1 mm. The number of apertures in each device may range from about 1 to about 1000 or more. Typically, the number of apertures in each dosage form ranges from about 5 to about 300. Most preferably, there are about 20 to about 200 apertures.

The apertures may be made by permanently removing tablet coating material of the appropriate size using either a mechanical, laser-based, or ultrasonic excitation process or other known techniques. Most preferably, a pulsed laser marking system is used to create the holes required. This system allows for an array of apertures to be created on both faces of a dosage form and at rates suitable for production of dosage forms.

This process utilizes a digitally controlled laser marking system (such as those manufactured by The Automation Partnership, Cambridge UK) to produce a programmable number of holes completely through the surface or coating of the dosage form, at rates practically suitable for production of dosage forms.

The steps involved in this laser drilling process are as follows: a pulsed laser marking system is focused at a tablet handling stage; the dosage form is moved by the tablet handling stage into the area of focused radiation created by the laser; the laser marking system is pulsed to provide sufficient power needed to remove areas of coating along a linear array on the dosage form; the dosage form is moved forward on the tablet handling stage; and the laser system is again pulsed as needed to produce additional linear arrays of apertures as necessary. The dosage form continues to be advanced by the tablet handling stage until it is eventually ejected from the system.

In one embodiment of the instant invention, a preferred coating comprises ten parts by weight of cellulose acetate butyrate and one part by weight of triethyl citrate dissolved in a mixture of acetone and ethanol (about 3:1 v/v). This mixture is sprayed on the core or dipped into the mixture so that a coating thickness of about 50 to about 250 microns is applied. More preferably, the thickness is about 80 to about 120 microns. Most preferably, the thickness is about 90 to about 110 microns. Another preferred coating for the impermeable wall may include: a mixture of eight parts by weight of cellulose acetate butyrate, two parts by weight of cellulose acetate and one part by weight of diethylphthalate. This mixture is dissolved in a solution of methylene chloride and methanol (about 3:1 v/v) and sprayed onto the cores to a thickness of about 100 to about 500 microns. Preferably, the thickness is about 200 to about 300 microns.

Coloring agents may be added to increase or decrease the absorption of the laser energy being utilized. Suspending agents may be added to the coating solution if the coloring agent being used is insoluble. Types of suspending agents include, but are not limited to, talc and titanium dioxide.

The polymers used in the coating which are herein described are known to the art or can be prepared according to the procedures in the Encyclopedia of Polymer Science and Technology, Vol. 3, published by Interscience Publishers, Inc., New York, in Handbook of Common Polymers by Scott, J. R. and Roff, W. J., 1971, published by CRC Press, Cleveland, Ohio.

In an embodiment of the instant invention, a film coating is applied prior to the application of the water insoluble, water impermeable polymeric coating. This film coating protects the formulation, such as a tablet, from attrition during the application of the polymeric coating. Preferably, the film coating comprises hydroxypropyl methylcellulose and hydroxypropyl cellulose.

In operation, the delivery device is ingested by a mammal and is contacted by the fluids in the environment of use (i.e. gastrointestinal tract). These fluids enter the delivery device through the apertures in the coating and hydrate the water swellable polymer and the pH modulator. Once the pH modulator dissolves, it maintains a sufficiently high pH level inside the core so that the beneficial agent remains insoluble and enhances the conditions for the swelling of the polymer. As the polymer swells, it moves some of the beneficial agent from the core of the device into the environment of use. This dispersion continues as the polymer swells to maximum capacity inside the delivery device. The swelling of the polymer is regulated by the pH modulator, which regulates hydration inside the core. Because small amounts of beneficial agent are released into a large volume of fluids (e.g. gastrointestinal system) over a period of time, the beneficial agent is easily dissolved, regardless of the pH of the environment of use. Thus, this device allows the delivery of a beneficial agent without relying on the pH of the environment of use to cause the release.

The drug delivery device of the instant invention may also be co-administered with other well known therapeutic agents that are selected for their particular usefulness against the condition that is being treated. For example, the instant invention may also be co-administered with other well known cancer therapeutic agents that are selected for their particular usefulness against the condition that is being treated. Included in such combinations of therapeutic agents are combinations of a prenyl-protein transferase inhibitors and an antineoplastic agent. It is also understood that such a combination of antineoplastic agent and inhibitor of prenyl-protein transferase may be used in conjunction with other methods of treating cancer and/or tumors, including radiation therapy and surgery.

Examples of an antineoplastic agent include, in general, microtubule-stabilizing agents (such as paclitaxel (also known as Taxol®), docetaxel (also known as Taxotere®), epothilone A, epothilone B, desoxyepothilone A, desoxyepothilone B or their derivatives); microtubule-disruptor agents; alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, trastuzumab (Herceptin™), 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

The preferred class of antineoplastic agents is the taxanes and the preferred antineoplastic agent is paclitaxel.

Radiation therapy, including x-rays or gamma rays which are delivered from either an externally applied beam or by implantation of tiny radioactive sources, may also be used in combination with the instant invention to treat cancer.

Additionally, the instant invention may also be useful for administering inhibitors of prenyl-protein transferase, which may be used as radiation sensitizers, as described in WO 97/38697, published on Oct. 23, 1997, and herein incorporated by reference.

The following examples illustrate the preparation of the drug delivery device of this invention and their sustained release of one or more therapeutically beneficial agents into an environment of use and as such are not to be considered as limiting the invention set forth in the claims appended hereto.

### EXAMPLES

Examples provided are intended to assist in a further understanding of the invention. Particular materials employed, species and conditions are intended to be further illustrative of the invention and not limitative of the reasonable scope thereof.

In the following example the farnesyl transferase inhibitor, 1-(3-chlorophenyl)-4-[1-(4-cyanobenzyl)-5-imidazolyl methyl]-2-piperazinone (as described in U.S. Pat. No. 5,856,326 and incorporated herein by reference), hereafter “the drug” or “beneficial agent”, is used as the model drug. The preparation of 1-(3-Chlorophenyl)-4-[1-(4-cyanobenzyl)-5-imidazolyl methyl]-2-piperazinone is also described in the following examples.

### Example 1

### Preparation of p-Cyanobenzylamine.H₃PO₄ Salt

A slurry of HMTA in 2.5 L EtOH was added gradually over about 30 min to about 60 min to a stirred slurry of cyanobenzyl-bromide in 3.5 L EtOH and maintained at about 48-53° C. with heating & cooling in a 22L neck flask (small exotherm). Then the transfer of HMTA to the reaction mixture was completed with the use of 1.0 L EtOH. The reaction mixture was heated to about 68-73° C. and aged at about 68-73° C. for about 90 min. The reaction mixture was a slurry containing a granular precipitate which quickly settled when stirring stopped.

The mixture was cooled to a temperature of about 50° C. to about 55° C. Propionic acid was added to the mixture and the mixture was heated and maintained at a temperature of about 50° C. to about 55° C. Phosphoric acid was gradually added over about 5 min to about 10 min, maintaining the reaction mixture below about 65° C. to form a precipitate-containing mixture. Then the mixture was gradually warmed to about 65° C. to about 70° C. over about 30 min and aged at about 65° C. to about 70° C. for about 30 min. The mixture was then gradually cooled to about 20-25° C. over about 1 hour and aged at about 20-25° C. for about 1 hour.

The reaction slurry was then filtered. The filter cake was washed four times with EtOH, using the following sequence, 2.5 L each time. The filter cake was then washed with water five times, using 300 mL each time. Finally, the filter cake was washed twice with MeCN (1.0 L each time) and the above identified compound was obtained.

### Example 2

### Preparation of 4-Cyanobenzylamine Hydrochloride via Hexamethylene-tetrammonium Salt

A 72 liter vessel was charged with 190 proof ethanol (14.4 L) followed by the addition of 4-cyanobenzylbromide (2.98 kg) and HMTA (2.18 kg) at ambient temperature. The mixture was heated to about 72-75° C. over about 60 min. On warming, the solution thickens and additional ethanol (1.0 liter) was added to facilitate stirring. The batch was aged at about 72-75° C. for about 30 min.

The mixture was allowed to cool to about 20° C. over about 60 min, and HCl gas (2.20 kg) was sparged into the slurry over about 4 hours during which time the temperature rose to about 65° C. The mixture was heated to about 70-72° C. and aged for about 1 hour. The slurry was cooled to about 30° C. and ethyl acetate (22.3 L) added over about 30 min. The slurry was cooled to about −5° C. over about 40 min and aged at about 3 to about 5° C. for about 30 min. The mixture was filtered and the crystalline solid was washed with chilled ethyl acetate (3×3 L). The solid was dried under a N₂ stream for about 1 hour before charging to a 50 liter vessel containing water (5.5 L). The pH was adjusted to about 10-10.5 with 50% NaOH (4.0 kg) maintaining the internal temperature below about 30° C. At about 25° C., methylene chloride (2.8 L) was added and stirring continued for about 15 min. The layers were allowed to settle and the lower organic layer was removed. The aqueous layer was extracted with methylene chloride (2×2.2 L). The combined organic layers were dried over potassium carbonate (650 g). The carbonate was removed via filtration and the filtrate concentrated in vacuo at about 25° C. to give a free base as a yellow oil.

The oil was transferred to a 50 liter vessel with the aid of ethanol (1.8 L). Ethyl acetate (4.1 L) was added at about 25° C. The solution was cooled to about 15° C. and HCl gas (600 g) was sparged in over about 3 hours, while keeping batch temperature below about 40° C. At about 20-25° C., ethyl acetate (5.8 L) was added to the slurry, followed by cooling to about −5° C. over about 1 hour. The slurry was aged at about −5° C. for about 1 hour and the solids isolated via filtration. The cake was washed with a chilled mixture of EtOAc/EtOH (9:1 v/v) (1×3.8 L), then the cake was washed with chilled EtOAc (2×3.8 L). The solids were dried in vacuo at about 25° C. to provide the above-titled compound.

¹H NMR (250 MHz, CDCl₃) δ7.83-7.79 (d, 2H), 7.60-7.57 (d, 2H), 4.79 (s, 2H), 4.25 (s, 2H); ¹³C NMR (62.9 MHz, CDCl₃) δ149.9, 139.8, 134.2, 131.2, 119.7, 113.4, 49.9, 49.5, 49.2, 48.8, 48.5, 48.2, 43.8.

### Example 3

### Preparation of 1-(4-Cyanobenzyl)-2-Mercapto-5-Hydroxymethylimidazole

7% water in acetonitrile (50 mL) was added to a 250 mL roundbottom flask. Next, an amine phosphate salt (12.49 g), as described in Example 2, was added to the flask. Next potassium thiocyanate (6.04 g) and dihydroxyacetone (5.61 g) was added. Lastly, propionic acid (10.0 mL) was added. Acetonitrilelwater 93:7 (25 mL) was used to rinse down the sides of the flask. This mixture was then heated to 6020 C., aged for about 30 minutes and seeded with 1% thioimidazole. The mixture was then aged for about 1.5 to about 2 hours at 60° C. Next, the mixture was heated to 70° C., and aged for 2 hours. The temperature of the mixture was then cooled to room temperature and was aged overnight. The thioimidazole product was obtained by vacuum filtration. The filter cake was washed four times acetonitrile (25 mL each time) until the filtrates became nearly colorless. Then the filter cake was washed three times with water (approximately 25-50 mL each time) and dried in vacuo to obtain the above-identified compound.

### Example 4

### Preparation of 1-(4-Cyanobenzyl)-5-Hydroxymethylimidazole

A 1 L flask with cooling/heating jacket and glass stirrer (Lab-Max) was charged with water (200 mL) at 25° C. The thioimidazole (90.27 g), as described in Example 3, was added, followed by acetic acid (120 mL) and water (50 mL) to form a pale pink slurry. The reaction was warmed to 40° C. over 10 minutes. Hydrogen peroxide (90.0 g) was added slowly over 2 hours by automatic pump maintaining a temperature of 35-45° C. The temperature was lowered to 25° C. and the solution aged for 1 hour.

The solution was cooled to 20° C. and quenched by slowly adding 20% aqueous Na2SO3 (25 mL) maintaining the temperature at less than 25° C. The solution was filtered through a bed of DARCO G-60 (9.0 g) over a bed of SolkaFlok (1.9 g) in a sintered glass funnel. The bed was washed with 25 mL of 10% acetic acid in water.

The combined filtrates were cooled to 15° C. and a 25% aqueous ammonia was added over a 30 minute period, maintaining the temperature below 25° C., to a pH of 9.3. The yellowish slurry was aged overnight at 23° C. (room temperature). The solids were isolated via vacuum filtration. The cake (100 mL wet volume) was washed with 2×250 mL 5% ammonia (25%) in water, followed by 100 mL of ethyl acetate. The wet cake was dried with vacuum/N₂ flow and the above-titled compound was obtained.

¹H NMR (250 MHz, CDCl₃): δ7.84-7.72 (d, 2H), 7.31-7.28 (d, 2H), 6.85 (s, 1H), 5.34 (s, 2H), 5.14-5.11 (t, 1H), 4.30-4.28 (d, 2H), 3.35 (s, 1H).

### Example 5

### Preparation of 1-(4-cyanobenzyl)-5-chloromethyl imidazole HCl Salt

1-(4-Cyanobenzyl)-5-hydroxymethylimidazole (1.0 kg), as described above in Example 4, was slurried with DMF (4.8 L) at 22° C. and then cooled to −5° C. Thionyl chloride (390 mL) was added dropwise over 60 min during which time the reaction temperature rose to a maximum of 9° C. The solution became nearly homogeneous before the product began to precipitate from solution. The slurry was warmed to 26° C. and aged for 1 h.

The slurry was then cooled to 5° C. and 2-propanol (120 mL) was added dropwise, followed by the addition of ethyl acetate (4.8 L). The slurry was aged at 5° C. for 1 h before the solids were isolated and washed with chilled ethyl acetate (3×1 L). The product was dried in vacuo at 40° C. overnight to provide the above-titled compound.

¹H NMR (250 MHz DMSO-d₆): δ9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

### Example 6

### Preparation of 1-(4-Cyanobenzyl)-5-Chloromethyl Imidazole HCl Salt via Addition of Hydroxymethylimidazole to Vilsmeier Reagent

To an ice cold solution of dry acetonitrile (3.2 L, 15 L/Kg hydroxymethylimidazole) was added 99% oxalyl chloride (101 mL, 1.15 mol, 1.15 equiv.), followed by dry DMF (178 mL, 2.30 mol, 2.30 equiv.), at which time vigorous evolution of gas was observed. After stirring for about 5 to 10 min following the addition of DMF, solid hydroxymethyl-imidazole (213 g, 1.00 mol), as described above in Example 4, was added gradually. After the addition, the internal temperature was allowed to warm to a temperature of about 23° C. to about 25° C. and stirred for about 1 to 3 hours. The mixture was filtered, then washed with dry acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded the crystalline form of the chloro-methylimidazole hydrochloride.

¹H NMR (250 MHz DMSO-d₆): δ9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

### Example 7

### Preparation of 1-(4-Cyanobenzyl)-5-Chloromethyl Imidazole HCl Salt via Addition of Vilsmeier Reagent to Hydroxymethylimidazole

To an ice cold solution of dry DMF (178 mL, 2.30 mol, 2.30 equiv.) in dry acetonitrile (2.56 L, 12 L/Kg Hydroxymethylimidazole) was added oxalyl chloride (101 mL, 1.15 mol, 1.15 equiv). The heterogeneous mixture in the reagent vessel was then transferred to a mixture of hydroxymethylimidazole (213 g, 1.00 mol), as described above in Example 4, in dry acetonitrile (1.7 L, 8 L/Kg hydroxymethylimidazole). Additional dry acetonitrile (1.1-2.3 L, 5-11 L/Kg hydroxymethylimidazole) was added to the remaining solid Vilsmeier reagent in the reagent vessel. This, now nearly homogenous, solution was transferred to the reaction vessel at T₁2+6° C. The reaction vessel temperature was warmed to a temperature of about 23° C. to about 25° C. and stirred for about 1 to 3 hours. The mixture was then cooled to 0° C. and aged 1 h. The solid was filtered and washed with dry, ice cold acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded the crystalline form of the chloromethylimidazole hydrochloride.

### Example 8

### Preparation of the Amide Alcohol

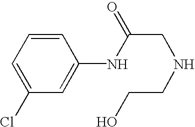

At 22° C., 3-chloroaniline (50.0 g) was combined with 460 ml isopropyl acetate and 20% aqueous potassium bicarbonate (72.5 g dissolved in 290 ml water). The biphasic mixture was cooled to 5° C. and chloroacetyl chloride (42 ml) was added dropwise over 30 minutes, keeping the internal temperature below 10° C. The reaction mixture was warmed to 22° C. over 30 min. The aqueous layer was removed at 22° C. and ethanolamine (92 ml) was added rapidly. The reaction mixture was warmed to 55° C. over 30 minutes and aged for 1 hour. At 55° C., 140 ml water was added with 30 ml isopropyl acetate to the reaction mixture. The biphasic reaction mixture was agitated for 15 minutes at 55° C. The layers were allowed to settle and the aqueous layer was removed. The organic layer was cooled to 45° C. and seed was added. The mixture was cooled to 0° C. over 1 hour and aged for 1 hour. The solids were filtered and washed with chilled isopropyl acetate (2×75 ml). The solids were dried in vacuo at 40° C. for 18 hours to provide the above-identified amide alcohol.

¹H NMR (300 MHz; DMSO-d₆) δ7.85 (t, 1H 2.0 Hz), 7.52 (m, 1H), 7.32 (t, 1H, 8.0 Hz), 4.5-4.8 (br s, 1H), 3.47 (t, 1H, 5.5 Hz), 3.30 (s, 1H), 2.60 (t, 1H 5.0 Hz).

¹³C NMR (75.4 MHz; DMSO-d₆) δ_{c} 170.9, 140.1, 133.0, 130.3, 122.8 118.5, 117.5, 60.3, 52.7, 51.5.

### Example 9

### Synthesis of 1-(3-Chlorophenyl)-2-Piperazinone Hydrochloride With DPAD

An amide alcohol, as described above in Example 8, was slurried with THF (37 ml) at 22° C., followed by the addition of tributyl phosphine (8.7 ml). The mixture was cooled to 0° C. and the DPAD was added in portions over 15 min. The slurry was aged at 0-5° C. for 30 minutes, warmed to 25° C. and aged for 18 hours. The reaction mixture was filtered and the cake was washed with THF (2×25 ml). The filtrate was concentrated in vacuo at <35° C. and combined with 50 ml of 2-propanol. The solution was cooled to 5° C., seeded with authentic material and treated with ethanol HCl (2.6 ml; 8.4M solution) dropwise over 20 min. The resulting slurry was recooled to 10° C. and aged for 1 hour. The solids were isolated and the cake and flask rinsed with chilled 2-propanol (2×10 ml). The product was dried in vacuo at 40° C. for 18 hours to provide the above-titled compound.

¹H NMR (300 MHz; DMSO-d₆) δ10.24 (br s, 2H), 7.50-7.30 (m, 4H), 3.92 (t, 2H, 5.5 Hz), 3.84 (s, 2H), 3.51 (t, 5.5 Hz); ¹³C NMR (75.4 MHz; DMSO-d₆) δ_{c} 162.1, 142.6, 132.9, 130.7, 127.0, 126.1, 124.54, 46.1, 44.9, 39.8.

### Example 10

### Synthesis of 1-(3-Chlorophenyl)-2-Piperazinone Hydrochloride With DIAD

58 mL of EtOAc was charged to an N₂-purged flask. Tributylphosphine (28.3 mL, 113.8 mmol) was added, via syringe, and the solution was cooled to about −10° C. DIAD (22.4 mL, 113.8 mmol) was added dropwise over 30 minutes, maintaining the temperature at <0° C. The above mixture was cannulated into a slurry of an amide alcohol (20.0 g, 87.5 mmol), as described above in Example 8, in 117 mL EtOAc over 20 minutes, maintaining the temperature at <0° C. The reaction was warmed to room temperature over 25 minutes. 99% conversion was observed by LC assay. Water (0.55 mL) was then added, and the reaction was warmed to 40° C. The solution was seeded with 200 mg of authentic material, and 1.0 eq. HCl (4.0 N in abs. EtOH) was added dropwise over 2 hours. The slurry was cooled to 0° C. over 2 hours and aged at 0° C. for 1 hour. The mixture was filtered, and the cake was washed with chilled EtOAc (3×16 mL). The cake was dried in vacuo overnight at 40° C. to afford the above-titled compound.

### Example 11

### Preparation of 1-(3-chlorophenyl)-4-[1-(4-cyanobenzyl)-5-imidazolylmethyl]-2-piperazinone.H₂O

A 50 L four-neck flask, equipped with a mechanical stirrer, cooling bath, teflon-coated thermocouple, and nitrogen inlet was charged with 4.0 L of acetonitrile. Then 4-cyanobenzyl-chloromethylimidazole hydrochloride, as described in Example 6 or 7 (958 g, 3.36 mol), piperazinone hydrochloride, as described in Example 9 or 10, (883 g, 3.54 mol), and the remaining 1.25 L of acetonitrile were added to the flask at room temperature. Diisopropylethylamine (1.99 L, 11.4 mol) was added to the mixture. The bulk of the solid dissolved immediately upon addition of diisopropylethylamine, leaving a slightly turbid solution.

After stirring 30 min, the solution was cooled to 0° C. over 60 min. The solution was stirred 26 h at 0° C., then warmed to 20° C. over 20 min. Water (2 L) was added to the slightly turbid solution over 20 min. Authentic seed was added to 8 L of water, which was subsequently added to the solution over 70 min. Additional water (17 L) was added over 90 min, and the mixture was aged 60 min thereafter. The temperature throughout the addition and aging was from about 20° C. to about 22° C. The mixture was filtered through a polypropylene filter pot. The crystals were washed with 1:5 acetonitrile/water. The crystalline solid was dried by passage of nitrogen through the filter cake (36 h) to provide the above-titled compound.

¹³C NMR (62.9 MHz, CDCl₃): δ165.2, 142.7, 142.1, 139.4, 134.8, 133.0, 131.0, 130.2, 127.3, 127.1, 126.3, 126.0, 123.9, 118.1, 112.0, 57.7, 50.6, 49.9, 148.8, 148.3.

### Example 12

The formulation of the instant invention was prepared using 1-(3-chlorophenyl)-4-[1-(4-cyanobenzyl)-5-imidazolyl methyl]-2-piperazinone is used as the beneficial agent. The solubility of the beneficial agent decreases from about 1 g/mL at pH 1 to about 70 μg/mL at pH 7, with the solubility cliff occurring at about pH 3.5.

Tablets for the pH-insensitive sustained release of the beneficial agent were prepared with the following composition:

| | | Amount Per |
|---|---|---|
| Ingredient | | Tablet (mg) |
| CORE TABLET | free base monohydrate of 1-(3- | 104.4¹ |
| | chlorophenyl)-4-[1-(4-cyanobenzyl)-5- | |
| | imidazolyl methyl]-2-piperazinone | |
| | Carbopol ® 974P NF | 27.10 |
| | Sodium phosphate dibasic USP | 135.5 |
| | anhydrous | |
| | Magnesium Stearate, NF (Non- | 4.000 |
| | Bovine) | |
| FILM COAT | Hydroxypropyl Methylcellulose USP | 4.065 |
| | 6CPS | |
| | Hydroxypropyl cellulose LF NF | 4.065 |
| | W/<0.3% Silica | |
| | Water For Injection, USP | 73.17² |
| CAB COAT | Cellulose acetate butyrate | 19.80 |
| | (CAB 381-20) | |
| | Triethyl citrate PG/NF | 1.980 |
| | Acetone NF | 335.2² |
| | Ethyl alcohol 200 proof USP | 111.7² |
| | Total Tablet Weight | 300.91 |

¹ The monohydrate conversion factor is 1.044.
² Removed during processing.

The beneficial agent, Carbopol® 974P NF and sodium phosphate were thoroughly mixed in a Patterson-Kelly® V-shell blender and lubricated with half of the magnesium stearate. The lubricated blend was then dry granulated using a Freund® TF-Mini roller compactor. The compacted ribbons were milled using a Quadro® cone milled and subsequently lubricated with the remainder of the magnesium stearate in the V-shell blender. Tablet cores with a target weight of 271 mg were compressed on a Korsch® rotary tablet press using 0.25″×0.36″ caplet shaped tooling. The tablet cores were film-coated using an aqueous solution of Hydroxypropyl cellulose (HPC) and Hydroxypropyl Methyl-cellulose (HPMC) in a Freund® HCT mini side-vented pan coater. This film coating was applied in order to minimize tablet attrition during the next coating step.

The film-coated tablets were then charged to a Glatt® column coater fitted with a Wurster insert and coated using a solution of cellulose acetate butyrate and triethylcitrate dissolved in a 3:1 (v/v) mixture of acetone and ethanol. The thickness of the cellulose acetate butyrate/triethylcitrate coating was approximately 110 μm. Forty-four circular apertures of 0.4 mm diameter were laser drilled on each face of the tablets. The laser driller assembly was manufactured by The Automation Partnership, Part No. TAP-1771-01-008.

The in vitro release of the drug from the tablets prepared as above was determined using USP Apparatus II, paddle speed 75 rpm, at 37° C. in 900 ml of dissolution media with pH values 1.2, 1.7 and 6.8. The pH 1.2 and 1.7 media consisted of 0.1N HCl solution adjusted to the appropriate pH by the addition of sodium hydroxide. The pH 6.8 medium consisted of 0.7% sodium dodecyl sulfate in 10 mM phosphate buffer.

Description of Figures

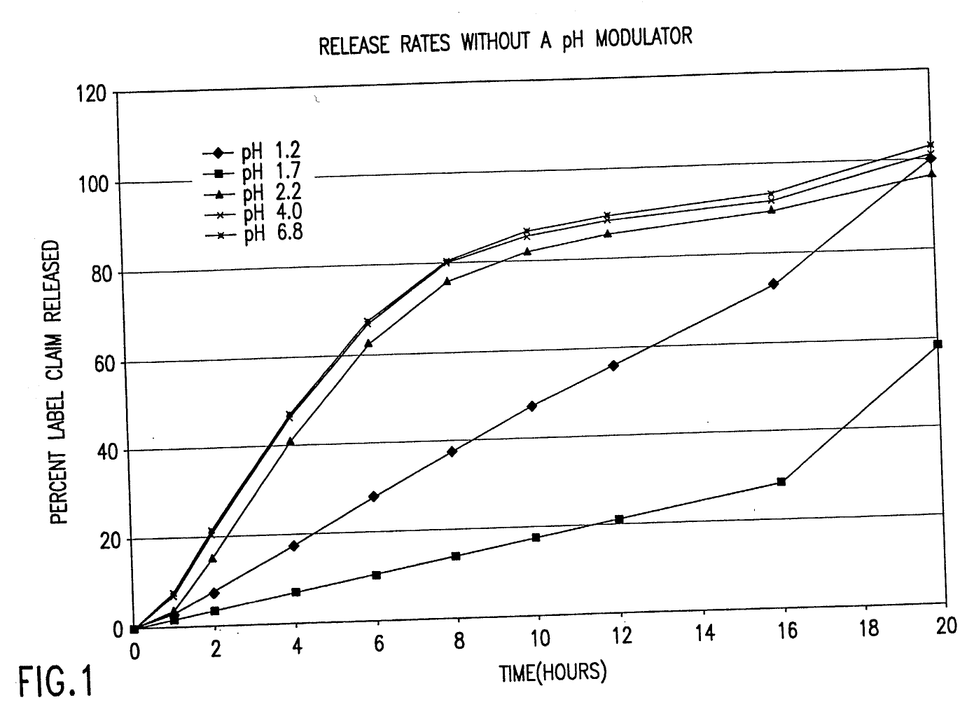

As shown in FIG. 1, the release rate of the beneficial agent at low pH levels (about 1.2), without a pH modulator, is dominated by dissolution and diffusion. Under strongly acidic conditions, the swelling of the polymer is greatly reduced but the high solubility of the beneficial agent results in release by dissolution/diffusion mechanism. At pH levels of about 1.7, both the dissolution/diffusion and gel extrusion mechanisms are repressed. The pH level in the core is too high for the beneficial agent to be appreciably soluble but it is also too low for the polymer to undergo sufficient ionization to result in gel extrusion of the beneficial agent. In media with pH levels between about 2.2 and about 6.8, the release of any beneficial agent is controlled by the gel extrusion mechanism. However, at such pH levels, the beneficial agent is poorly soluble.

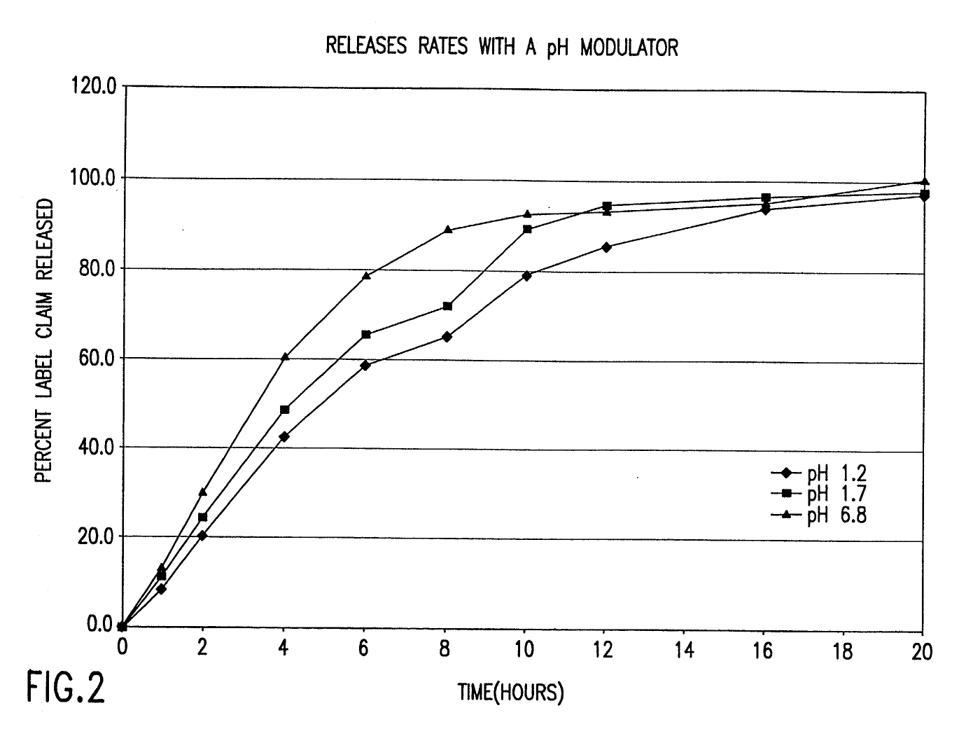

By adding a pH modulator, as shown in FIG. 2, the release rate of the beneficial agent is predominantly controlled by the gel extrusion mechanism and is relatively similar over a range of pH levels.

What is claimed is:

1. A drug delivery device, that is pH insensitive, for the sustained in situ production and release of a dispersion in an environment of use, which comprises
a) a compressed core prepared from an admixture comprising
i) a therapeutically effective amount of a beneficial agent that has a solubility profile that is dependent on the pH level of the environment of use;
ii) a water swellable polymer which upon hydration forms gelatinous microscopic particles; and
iii) a pH modulator; and
b) a water insoluble, water impermeable polymeric coating comprising a polymer and a plasticizer, which surrounds and adheres to the compressed core, said water insoluble, water impermeable polymeric coating having at least one aperture.

2. The device of claim 1, wherein the beneficial agent comprises a prenyl protein inhibitor.

3. The device of claim 1, wherein the beneficial agent comprises a farnesyl-protein transferase inhibitor.

4. The device of claim 3, wherein the beneficial agent is 1-(3-Chlorophenyl)-4-[1-(4-cyanobenzyl)-5-imidazolylmethyl]-2-piperazinone or its pharmaceutically acceptable salts or hydrates.

5. The device of claim 4, wherein the amount of beneficial agent in the core comprises from about 0.01% to about 75% by weight of the core mixture.

6. The device of claim 4, wherein the amount of swellable polymer in the core comprises from about 5% to about 75% by weight of the core mixture.

7. The device of claim 4, wherein the amount of pH modulator in the core comprises from about 1% to about 75% by weight of the core mixture.

8. The device of claim 7, wherein the amount of pH modulator in the core comprises from about 10% to about 65% by weight of the core mixture.

9. The device of claim 8, wherein the amount of pH modulator in the core comprises from about 40% to about 55% by weight of the core mixture.

10. The device of claim 4, wherein the pH modulator comprises bases, salts, sugars, surfactants or soluble polymers.

11. The device of claim 10, wherein the pH modulator comprises sodium citrate, betaine hydrochloride, sodium bicarbonate, sodium phosphate, sodium carbonate or arginine.

12. The device of claim 4, wherein the apertures in the coating range from about 0.05 mm to about 20 mm at their widest point.

13. The device of claim 12, wherein the apertures in the coating are arranged in a regular or irregular pattern about one or both of the surfaces of the device.

14. The device of claim 13, wherein the number of apertures in the coating range from about 1 to about 1000.

15. The device of claim 14, wherein the number of apertures in the coating range from about 20 to about 200.

16. The device of claim 4, wherein additional excipients may be added to the compressed core to neutralize the pH value of the environment of use.

17. A process for the preparation of the device of claim 1 for the sustained release of a beneficial agent which comprises:
a) preparing the compressed core by either dry or wet granulation of the swellable polymer, the medicament and other excipients required in the preparation of tablets and compressing the mixture into cores;
b) coating the entire core with the coating material; and
c) putting apertures through the coating using mechanical, laser-based, or ultrasonic excitation techniques.

18. A method of treating cancer with a therapeutically effective amount of a beneficial agent by administering the drug delivery device of claim 2 to a mammal in need thereof.

19. A method of conferring radiation sensitivity on a tumor cell using a therapeutically effective amount of a beneficial agent by administering the drug delivery device of claim 3 in combination with radiation therapy.

20. A method of treating cancer using a therapeutically effective amount of a beneficial agent by administering the drug delivery device of claim 2 in combination with an antineoplastic.

21. A method according to claim 20 wherein the antineoplastic is paclitaxel.
